(19) United States
(12) Patent Application Publication
(10) Pub. No.: US 2007/0032830 A1
(43) Pub. Date: Feb. 8, 2007

(54) Automatic external defibrillator (AED) with wireless communications

(76) Inventor: Kyle R. Bowers, Boxborough, MA (US)

Correspondence Address: Mark J. Pandiscio;Pandiscio & Pandiscio, P.C., 470 Totten Pond Road, Waltham, MA, 02451-1914, US

(21) Appl. No.: 11/499,273

(22) Filed: Aug. 4, 2006

Related U.S. Application Data
(60) Provisional application No. 60/705,351, filed on Aug. 4, 2005.

(51) Int. Cl. A61N 1/39

(52) U.S. Cl. 607/5

(57) ABSTRACT

An Automatic External Defibrillator (AED) with wireless communications contained within the device. The wireless system is used to contact a remote emergency specialist. The remote instructor guides the lay rescuer through the resuscitation effort, thereby increasing the likelihood for successful defibrillation.

### REFERENCE TO PENDING PRIOR PATENT APPLICATION

This patent application claims benefit of pending prior U.S. Provisional Patent Application Ser. No. 60/705,351, filed Aug. 4, 2005 by Kyle R. Bowers for AUTOMATIC EXTERNAL DEFIBRILLATOR WITH WIRELESS COMMUNICATIONS (Attorney's Docket No. ACCESS-8 PROV), which patent application is hereby incorporated herein by reference.

### FIELD OF THE INVENTION

This invention relates to Automatic External Defibrillators (AEDs) in general and, more particularly, to Automated External Defibrillators (AEDs) with wireless communications that are capable of contacting a remote emergency instructor for guiding a lay rescuer through a resuscitation event.

### BACKGROUND OF THE INVENTION

Approximately 350,000 deaths occur each year in the United States alone due to Sudden Cardiac Arrest (SCA). Worldwide deaths due to Sudden Cardiac Arrest (SCA) are believed to be at least twice that of the United States. Many of these deaths can be prevented if effective defibrillation is administered within 3-5 minutes of the onset of SCA.

Sudden Cardiac Arrest (SCA) is the onset of an abnormal heart rhythm, lack of pulse and absence of breath, leading to a loss of consciousness. If a pulse is not restored within a few minutes, death occurs. Most often, SCA is due to Ventricular Fibrillation (VF), which is a chaotic heart rhythm that causes an uncoordinated quivering of the heart muscle. The lack of coordinated heart muscle contractions results in a lack of blood flow to the brain and other organs. Unless this chaotic heart rhythm is quickly terminated, thereby allowing the heart to restore its own normal rhythm, death ensues.

Rapid defibrillation is the only known means to restore a normal heart rhythm and prevent death after SCA due to Ventricular Fibrillation (VF). For each minute that passes after the onset of SCA, mortality typically increases by 10%. At 7-10 minutes, the survival rate is generally below 10%. However, if a patient is effectively defibrillated within 1-2 minutes of the onset of SCA, survival rates can be as high as 90% or more. Therefore, the only known way to increase the chance of survival for an SCA victim is through early defibrillation.

Automatic External Defibrillators (AEDs) offer the prospect of such early defibrillation, but they must be (i) portable so they can be easily carried to an SCA victim, (ii) easy-to-use so that they can be properly utilized when SCA occurs, and (iii) easily maintained.

AED programs provide formal training to potential rescuers with respect to AED use and Cardiopulmonary Resuscitation (CPR). However, as AEDs become more widespread in homes, offices and public places (e.g., airports), the probability increases that people with little or no medical training (i.e., “lay” rescuers) will attempt to use these devices. In these emergency situations where time is critical, the rescuer may fail to use the device properly, or may not use the device at all. In addition, lack of CPR training further reduces the chances of an SCA victim's survival, since the application of CPR in conjunction with defibrillation helps maintain blood flow to the brain (and other organs) while normal cardiac rhythm is restored.

### SUMMARY OF THE INVENTION

AEDs are intentionally designed to be easy to use. In accordance with the present invention, it is believed that if simple guidance were available, a “lay” rescuer could be effectively directed through a successful rescue. Furthermore, and also in accordance with the present invention, it is believed that quick dispatch of Advance Life Support (ALS), combined with automated location identification, could further increase the chances of survival. The present invention provides these features, among others.

The present invention is an Automatic External Defibrillator (AED) that provides wireless communications within the device.

In accordance with one preferred form of the present invention, the new AED contains a set of electrodes that are applied directly to the patient from the defibrillator. The pads contain an electrically conductive hydrogel that adheres the patient's skin. The defibrillator uses the electrodes to sense ElectroCardioGram (ECG) signals from the patient so as to determine the condition of the patient's heart and hence identify a shockable or non-shockable condition. The defibrillator also uses the electrodes to sense the patient's transthoracic impedance so as to determine the appropriate shock parameters. If a shockable condition is indicated, the defibrillator applies a pulsed voltage potential at the electrodes, which causes a flow of electrical current through the patient's chest.

In accordance with one preferred form of the present invention, the AED contains a shock delivery circuit, which is used to deliver an appropriate biphasic shock to the patient.

In accordance with one preferred form of the present invention, the shock delivery circuit contains a battery, high voltage capacitors, a circuit to charge the capacitors from the battery, and a circuit to deliver a biphasic waveform from the capacitors to the patient.

In accordance with one preferred form of the present invention, the AED contains an ECG and impedance analysis circuit to determine if the patient requires therapy and to measure and analyze the patient's transthoracic impedance, so that the therapeutic waveform is delivered to the patient in a controlled and accurate manner.

In accordance with one preferred form of the present invention, the AED contains a circuit and antenna for wireless communications.

In accordance with one preferred form of the present invention, the AED is capable of contacting a remote medical specialist via the wireless communications.

In accordance with one preferred form of the present invention, the AED contains a user interface to facilitate interaction with the user and to guide the user through a sequence of rescue events.

In accordance with one preferred form of the present invention, the AED user interface provides buttons which may be used to control the device.

In accordance with one preferred form of the present invention, the AED user interface contains a microphone and speaker to transmit voice and/or other audio over the wireless communications.

In accordance with one preferred form of the present invention, the AED user interface contains a high-resolution Liquid Crystal Display (LCD), voice playback circuitry, an audio amplifier and a speaker, all of which may be used to guide the rescuer through a resuscitation effort.

In accordance with one preferred form of the present invention, the AED contains a controller circuit which operates the device.

In accordance with one preferred form of the present invention, the controller circuit contains one or more microprocessors, memory, and other circuitry to enable AED operation, including wireless communications.

### BRIEF DESCRIPTION OF THE DRAWINGS

FIG. 1 is a diagram of the new AED and its electrodes attached to the patient;

FIG. 2 is a schematic diagram showing a high-level system block diagram of the new AED;

FIG. 3 is a schematic diagram showing a more detailed block diagram of the new AED;

FIG. 4 is a diagram showing one example of the AED's device labeling;

FIG. 5 is a diagram showing an exemplary AED prompt to call a remote medical specialist;

FIG. 6 is a diagram showing one example of the AED's pre-programmed location;

FIG. 7 is a flow diagram showing how the new AED is used in a typical resuscitation effort;

FIG. 8 is a flow diagram showing an example of a remote emergency instruction protocol; and

FIG. 9 is an exemplary table of the patient condition codes transmitted by the new AED.

### DETAILED DESCRIPTION OF THE PREFERRED EMBODIMENT

In accordance with the present invention, the new AED is provided with wireless communications to allow the unit to contact a remote medical specialist. This permits the specialist to provide verbal instructions to the lay rescuer whereby to guide the lay user through a resuscitation effort, thereby ensuring that the SCA victim receives appropriate therapy in a timely manner, thus increasing the likelihood of a successful outcome.

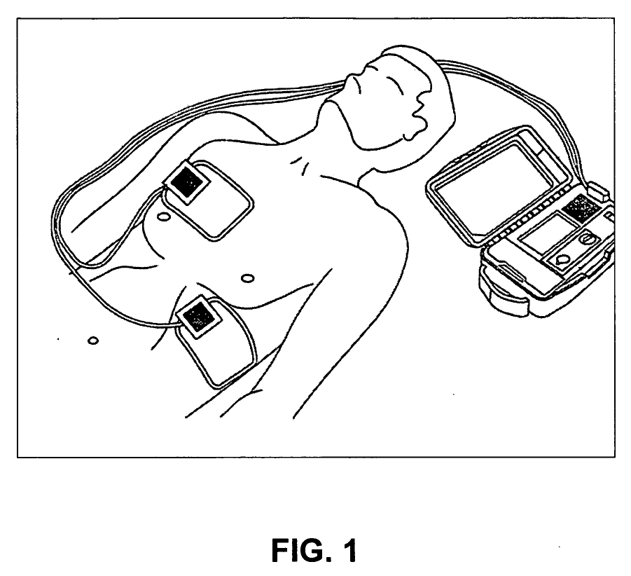

A typical connection of the AED to the patient is shown in FIG. 1.

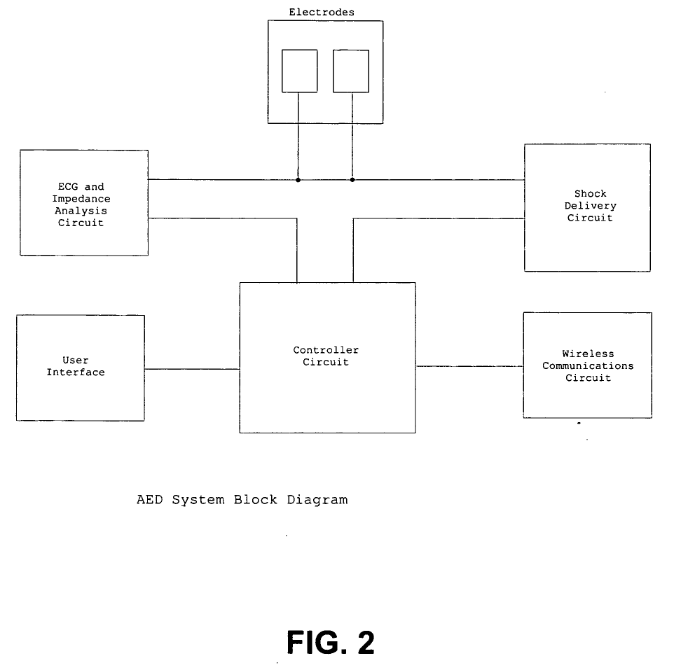

The present invention comprises an Automatic External Defibrillator (AED) that contains wireless communications within the device, as shown in the high level system block diagram in FIG. 2.

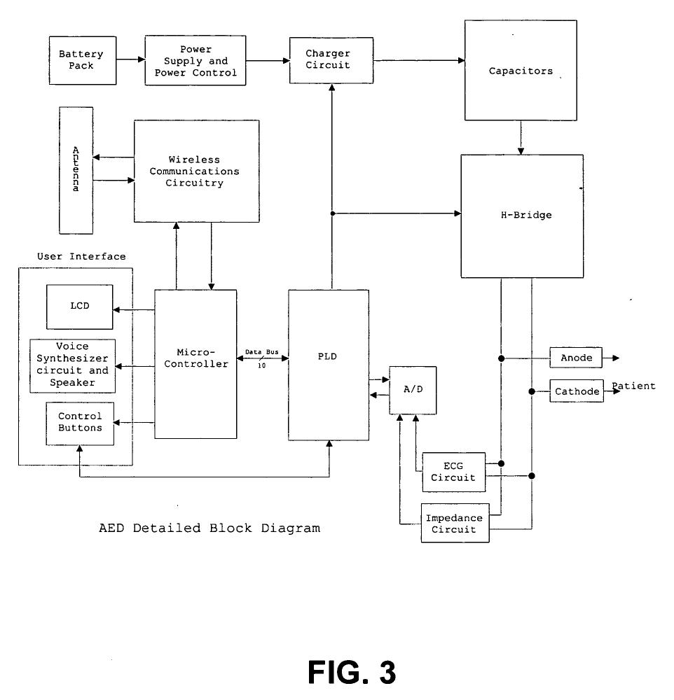

A more detailed block diagram of the new AED is shown in FIG. 3. The wireless communications apparatus is contained within the new AED. In a preferred embodiment of the present invention, the new AED contains a cell phone chipset that is capable of automatically dialing a remote emergency medical specialist. Once a two-way telephone connection is established, the specialist is able to guide the lay user through the resuscitation event.

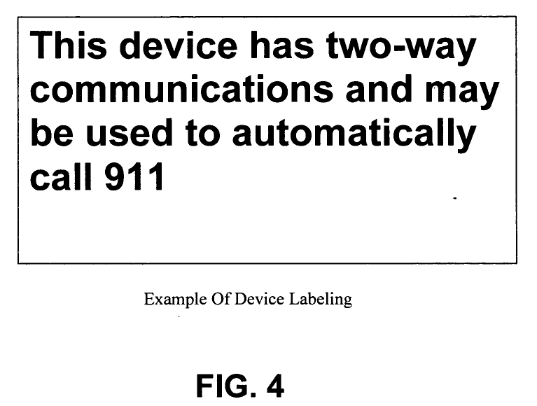

The new AED is labeled with information about its unique communications capability, so that the lay user is immediately alerted as to the communications capability of the device. An exemplary view of one such labeling is shown in FIG. 4.

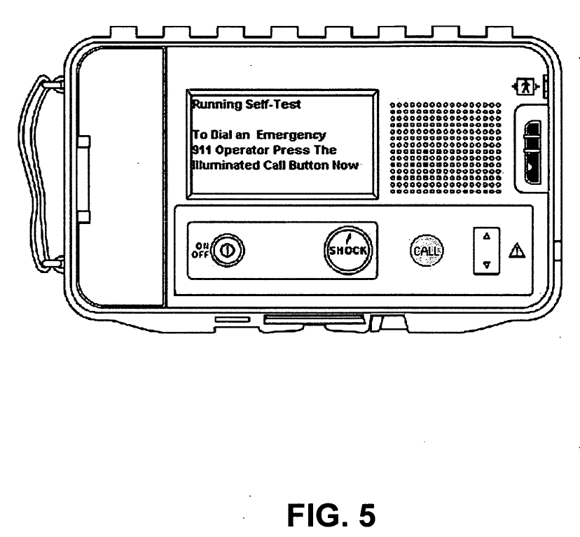

When the new AED is first powered on, it runs through self-test and then prompts the rescuer with the option of calling the emergency medical specialist. If the rescuer does not respond to this prompt within a predetermined period of time, the AED will automatically call the emergency specialist. In a preferred embodiment of the present invention, when the user wants to call the emergency medical specialist, the user simply presses a special “Call” button that is illuminated during the user prompt. An exemplary diagram showing the call button is shown in FIG. 5. The AED is configured so that the user may also call the emergency medical specialist at other periods of operation as well.

In a preferred embodiment of the present invention, the AED includes a pre-programmed call number that is used to contact the remote medical specialist.

In another preferred embodiment of the invention, the AED includes a prioritized list of numbers that are used in the event the higher priority number is busy, out-of-service or unreachable for other reasons. In other words, the AED automatically dials the highest priority number and, if it is unable to complete this call, proceeds to the next highest priority number, and continues in this fashion until a connection is completed or the list is exhausted, in which case the AED may return to the top of the list and repeat the process.

As those skilled in the art can appreciate, the remote medical specialist could be a specially trained 911 operator or special emergency service personnel, such as local fire, police or ambulance departments. Once the rescuer establishes communications with the emergency specialist, the rescuer identifies his or her precise location. The remote specialist can then dispatch emergency medical personnel to the patient's location.

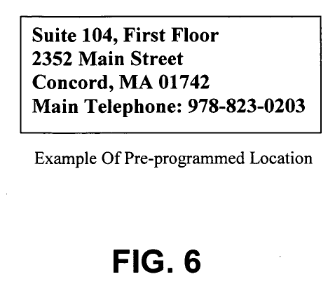

In another aspect of the invention, the AED can be pre-programmed with its location and, when the AED calls through to an emergency specialist, the AED can simultaneously transmit its pre-programmed location to the emergency specialist. The AED location may be programmed into the device (see FIG. 6) using the device setup menu.

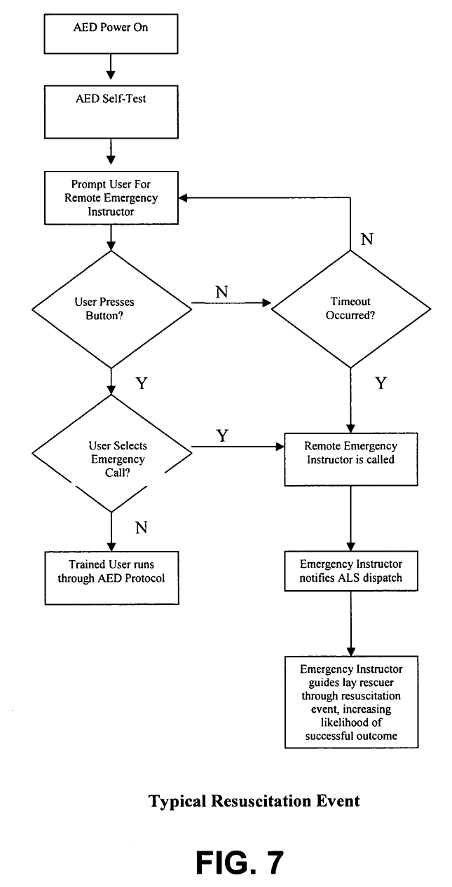

FIG. 7 is a flow diagram illustrating how the new AED may be used in a typical resuscitation effort.

In another aspect of the present invention, the AED may be provided with a Global Positioning System (GPS) unit. The GPS unit automatically identifies the location of the AED, eliminating the need to pre-program the AED with its location (or, alternatively, supplementing such location pre-programming, e.g., in case the AED is moved to a new location without appropriate reprogramming). The remote medical specialist receives the location of the AED as identified by the GPS unit and can direct emergency ALS dispatch appropriately.

In a preferred embodiment of the present invention, both the GPS and location pre-programming are provided, and the device is configured to that the pre-programmed location is transmitted if the GPS is not available or if the GPS is unable to locate the device.

In another preferred embodiment of the invention, the AED contains a Bluetooth chipset and uses a remote cell phone to call the remote emergency specialist.

In another preferred embodiment of the invention, the AED contains a Radio Frequency (RF) chipset, which communicates with an AED base station, which in turn contains a wired phone system to call the emergency specialist. By way of example, multiple AEDs might be deployed in an airport, with each of the AEDs communicating with a single base station, with the base station having a landline telephone connection.

In yet another aspect of the invention, the AED uses Wi-Fi, ZigBee or another wireless network to communicate with the base station. The base station might be connected to the emergency specialist via a landline, or the base station may be connected to the emergency specialist via the Internet.

In addition, the AED may be directly connected to the medical specialist via the Internet, without going through a base station.

In addition, the AED is capable of using voice-over-IP or other protocols as well.

The new AED also contains the necessary components for defibrillation including, but not limited to, a battery pack, capacitor charger circuit, high-voltage capacitors and an H-bridge circuit (see FIG. 3).

The defibrillator contains controllers for operating the defibrillator and wireless communications. These controllers may include microprocessors, microcontrollers, digital signal processors, field programmable gate arrays, programmable logic devices, and other digital or analog circuitry.

The defibrillator also contains several other components such as, but not limited to, a real time clock, analog-to-digital converters, digital-to-analog converters, operational amplifiers, audio amplifiers, random access memory, flash memory, EEPROM and other memories (both internal and removable).

The defibrillator also contains a high-resolution LCD screen, voice synthesizer circuit and speaker for instructing the rescuer during device use.

In one preferred embodiment of the present invention, the defibrillator LCD screen may be TFT or similar technology, capable of displaying high-resolution pictures and video.

The defibrillator also includes several buttons for user control. These buttons may comprise, but are not limited to, a power button, a shock button, a call button and special purpose buttons. These buttons can be seen on the device keypad as shown in FIG. 5.

In one preferred embodiment of the present invention, the defibrillator's wireless communications unit is configured to operate in full-duplex mode so as to simultaneously transmit and receive data.

In one preferred embodiment of the present invention, the defibrillator's wireless communication unit is capable of working over analog cell-phone systems such as Advanced Mobile Phone System (AMPS) or Narrowband Advanced Mobile Phone Service (NAMPS) using Frequency-Shift Keying (FSK) or other techniques to transmit and receive data.

In another preferred embodiment of the present invention, the defibrillator's wireless communications unit is capable of working in a multi-mode operation over analog, digital or mixed cell-phone systems using Frequency Division Multiple Access (FDMA), Time Division Multiple Access (TDMA), Code Division Multiple Access (CDMA), Wide Code Division Multiple Access (WCDMA) or other techniques to transmit and receive data.

In yet another preferred embodiment of the present invention, the defibrillator's wireless communications unit is capable of working over Personal Communications Services (PCS) systems.

In yet another preferred embodiment of the present invention, the defibrillator's wireless communications unit is capable of working over Universal Mobile Telephone System (UMTS) systems.

In yet another preferred embodiment of the present invention, the defibrillator's wireless communications unit is capable of working over Third Generation (3G) systems.

The new AED is very simple to operate. Once the pads are removed from their pouch and installed on the patient, the device automatically analyzes the patient's rhythm. If the device determines that the patient's rhythm is shockable, it charges the capacitors and notifies the user that a shock is advised and to stand clear from the patient. Once the device is ready, the shock button is illuminated and the lay rescuer simply presses the button to deliver a shock. The AED senses the patient's impedance, determines the appropriate shock parameters, and then delivers the therapeutic shock. FIG. 1 is a pictorial diagram of the AED applied to the patient.

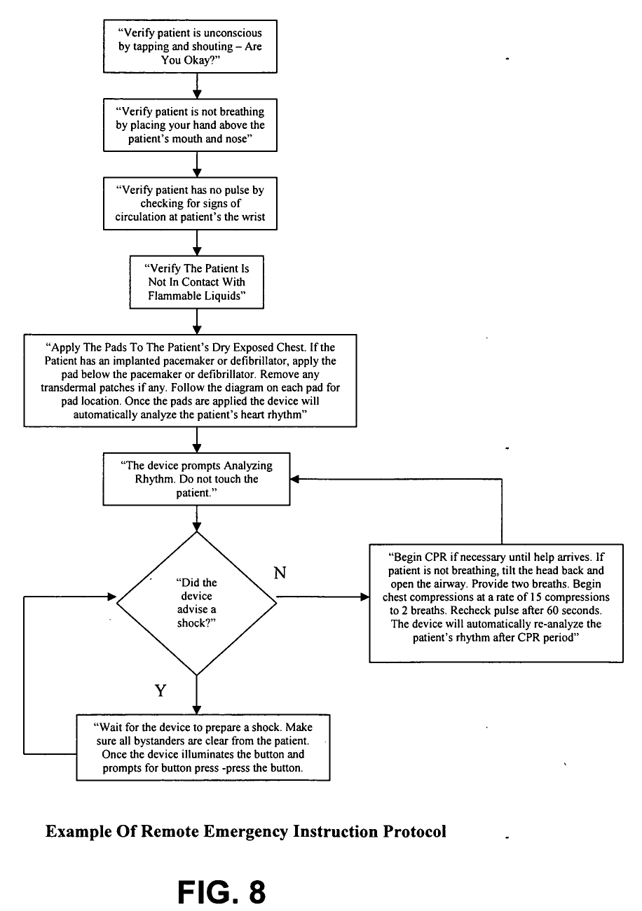

The remote emergency specialist has formal AED, CPR and emergency medical training. An example of the protocol used to guide the lay rescuer is shown in FIG. 8. In some ways the protocol is similar to the device prompts. However, lay rescuers may not be able to follow the prompts in emergency situations.

The remote specialist can also use the AED's communications unit to guide the lay rescuer through other scenarios as well, such as bleeding, burns, drowning, etc. The specialist also utilizes the communications unit to notify the lay rescuer when help is arriving and to intercept the dispatch when the exact location is not known.

In a preferred embodiment of the present invention, the new AED transmits data over the wireless communications; this transmitted data is received, decoded and displayed to the medical specialist so that the specialist can determine the appropriate treatment for the patient.

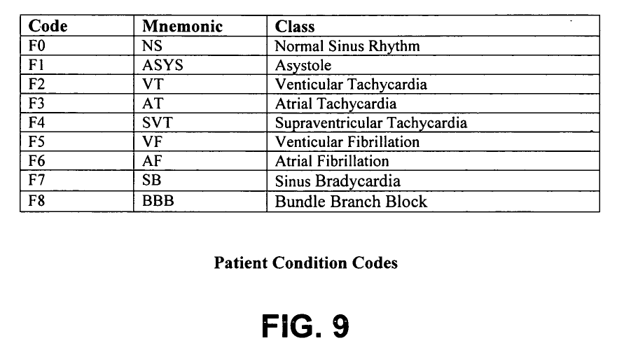

In one preferred embodiment of the present invention, the transmitted data consists of codes that indicate the patient's cardiac condition. One example of these codes is shown in FIG. 9.

In yet another preferred embodiment of the present invention, the transmitted data consists of the patient's ECG, which is displayed in real time, so the trained instructor can interpret the rhythm and determine the appropriate treatment for the patient.

As those skilled in the art will appreciate, the present invention may also be modified to include other types of sensors, such as respiratory, pulse-oximetry and non-invasive blood pressure. The data from these sensors could also be transmitted and displayed for the remote medical specialist.

The AED's two-way communications unit allows for an interactive resuscitation effort. In special situations the medical specialist can guide the lay rescuer through a customized protocol. The specialist is capable of asking numerous questions to assess the condition of the patient. This affords information gathering to begin prior to the arrival of rescue team. For example, if a patient has existing medical conditions or is on prescribed medications, the rescuer can communicate such facts to the remote medical specialist. This information is also relayed to the ALS dispatch being sent out to the victim and, in some cases, to the hospital awaiting arrival of the patient, so that appropriate preparations can be made to treat the patient. In addition, the lay rescuer may also request information from the remote specialist during the resuscitation effort.

In a preferred embodiment of the present invention, the AED may receive graphical instructions from the specialist to be displayed to the rescuer on the LCD screen.

In another preferred embodiment of the present invention, the defibrillator may receive graphical instructions in the form of pictures and/or video from the remote specialist. The graphical instructions may show, for example, how to clear a victim's blocked airway before giving breaths.

The AED with wireless communications greatly increases the likelihood of success in treating a victim of SCA.

While the present invention has been described in terms of certain exemplary preferred embodiments, it will be readily understood and appreciated by those skilled in the art that it is not so limited, and that many additions, deletions and modifications may be made to the preferred embodiments discussed herein without departing from the scope of the invention.

What is claimed is:

1. An automatic external defibrillator (AED) comprising:
at least one battery;
at least one capacitor;
a charging circuit to charge the at least one capacitor from the at least one battery;
a pair of electrodes for attachment to a patient;
a shock delivery circuit to deliver energy from the at least one capacitor to the patient through the electrodes, the shock delivery circuit being configured to deliver the shock to the patient in a biphasic waveform; and
a wireless communications unit for communicating with a remote site.

2. An automatic external defibrillator (AED) according to claim 1 wherein the wireless communications unit comprises a microphone and a speaker for two-way audio communications.

3. An automatic external defibrillator (AED) according to claim 1 wherein the wireless communications unit comprises data communications.

4. An automatic external defibrillator (AED) according to claim 3 wherein the data represents the patient's cardiac condition.

5. An automatic external defibrillator (AED) according to claim 3 wherein the data represents the patient's ECG signal.

6. An automatic external defibrillator (AED) according to claim 3 wherein the data comprises graphics.

7. An automatic external defibrillator (AED) according to claim 3 wherein the data comprises pictures.

8. An automatic external defibrillator (AED) according to claim 3 wherein the data comprises video.

9. An automatic external defibrillator (AED) according to claim 1 wherein the wireless communications unit operates in full-duplex mode to simultaneously transmit and receive.

10. An automatic external defibrillator (AED) according to claim 1 wherein the wireless communications unit is configured to call a remote emergency specialist.

11. An automatic external defibrillator (AED) according to claim 10 wherein the automated external defibrillator (AED) automatically calls the remote emergency specialist after a predetermined period of time.

12. An automatic external defibrillator (AED) according to claim 10 wherein a pre-programmed call number is used to contact the remote specialist.

13. An automatic external defibrillator (AED) according to claim 12 wherein a pre-programmed, prioritized list of phone numbers is used to call the remote specialist.

14. An automatic external defibrillator (AED) according to claim 10 wherein data is transmitted to the remote specialist.

15. An automatic external defibrillator (AED) according to claim 10 wherein the wireless communications unit is used to contact an AED base station, which in turn calls the remote emergency specialist.

16. An automatic external defibrillator (AED) according to claim 1 wherein the automatic external defibrillator is configured to report its location to a remote site.

17. An automatic external defibrillator (AED) according to claim 16 wherein the automated external defibrillator (AED) comprises a GPS unit for identifying the location of the device.

18. An automatic external defibrillator (AED) according to claim 16 wherein the automated external defibrillator (AED) is pre-programmed with its location.

19. An automatic external defibrillator (AED) according to claim 16 wherein the automated external defibrillator (AED) further comprises a GPS unit for identifying the location of the device, and wherein the device is also pre-programmed with its location, and further wherein the user defined pre-programmed location is used to locate the device if the GPS unit is unable to determine the device location.

20. An automatic external defibrillator (AED) according to claim 1 wherein the device further comprises user interface controls.

21. An automatic external defibrillator (AED) according to claim 20 wherein the user interface controls comprise an LCD display, voice playback circuitry, an audio amplifier and a speaker.

22. An automatic external defibrillator (AED) according to claim 20 wherein the device uses the user interface controls to provide the rescuer with a prompt to call the remote emergency specialist.

23. An automatic external defibrillator (AED) according to claim 20 wherein a call button is used to initiate communications with a remote specialist.
